(19) United States
(12) Patent Application Publication
(10) Pub. No.: US 2006/0073529 A1
(43) Pub. Date: Apr. 6, 2006

(54) Luciferyl peptide substrate

(76) Inventors: Christopher H. Contag, San Jose, CA (US); Rajesh R. Shinde, Sunnyvale, CA (US); Mark D. Bednarski, Lushing, MI (US); Samira Guccione, Hillsborough, CA (US)

Correspondence Address: REED INTELLECTUAL PROPERTY LAW GROUP, 1400 PAGE MILL ROAD, PALO ALTO, CA, 94304-1124, US

(21) Appl. No.: 11/204,480

(22) Filed: Aug. 15, 2005

Related U.S. Application Data
(60) Provisional application No. 60/601,597, filed on Aug. 13, 2004.

(51) Int. Cl. G01N 33/574; C12Q 1/66; C07K 7/06

(52) U.S. Cl. 435/7.23; 435/8; 530/330

(57) ABSTRACT

Provided are luciferyl peptide substrates that are produced by attaching specifically prepared peptide conjugates to luciferin, and/or its analogs and derivatives. The luciferyl peptide substrates are incapable of penetrating cell membranes and tissue barriers. Cleavage of the peptide conjugates from the luciferyl peptide substrates releases the luciferin, which upon contact with luciferase emits photons for easy detection. The luciferyl peptide substrates may be used in assays to detect pathogens, test protease inhibitors, probe cell physiology, assess protease activity in oncogenesis, and improve specific and regulated drug delivery.

### TECHNICAL FIELD

The invention relates generally to the field of multifunctional bioluminescent substrates. More specifically, the invention relates to the preparation of luciferyl peptide substrates from luciferin, its analogs, and derivatives combined with specifically prepared peptides, and the use of the luciferyl peptide substrates in improved assays to detect pathogens, test protease inhibitors, probe cell physiology, assess protease activity in oncogenesis, and improve specific and regulated drug delivery.

### BACKGROUND OF THE INVENTION

Biological processes can be studied both in vitro and in vivo. In vitro methods provide the ability to isolate molecules from the complex milieu of a biological system. Thus, they provide simpler settings for studying individual reactions than can be found in vivo. On the other hand, in vitro reactions do not usually take into account the effects of the surrounding systems on the particular reaction being studied. In vivo studies are much more complex, as multiple systems may be affecting the reaction being studied, making it difficult to identify the reaction and its parameters within the systems. However, studying a biological process in its native setting provides a more accurate picture of the process as it occurs in an organism.

Biological imaging in vivo is a preferred method for studying biological processes, as it provides access to information in real time. It allows monitoring of such parameters as location, procedure, and time. Of particular interest, non-invasive imaging methods allow continuous, undisturbed monitoring. To avoid use of repeated, time-stacked, sacrifice of animals, reporter systems have been developed that can be followed extra-corporeally. Examples of effective reporter systems include radiolabeled probes, enzyme-linked probes, and luminescent and fluorescent probes.

As noted in Contag et al., ANN. REV. BIOMED. ENG. 4:235-260 (2002), three important elements that need to be present in a biological imaging system are (a) longevity of the marker/probe system so that a process can be studied over the full study period, (b) sensitivity of the markers and probes to very small changes in the system being studied, and (c) ability of the markers and probes not to interfere with the system being monitored.

Fluorescent markers have been used with reasonable success. Drawbacks of using fluorescent markers include the requirement to stimulate the markers by an energy source outside the tissue or animal being studied. This can cause scatter and low efficiency of the marker. Additionally, some markers, such as the green fluorescent protein markers, emit in the wavelength range that is absorbed and quenched by tissue. Interference from autofluorescence from endogenous molecules, such as hemoglobin and cytochromes, can also decrease the ability to detect such markers.

Bioluminescent markers remove the requirement for an outside energy source. Luciferases, which are found in certain bacteria, marine crustaceans, fish, and insects, are luminescent enzymes that utilize oxygen, and often ATP, as energy sources, causing luminescence in the visible light range upon interaction with luciferin or related molecules. The wide variety of luciferases provides markers that emit in the range of 460-630 nm. Ibid. In particular, the luciferase from the North American firefly Photinus pyralis has been used as a marker. Its gene, luc, has been cloned and modified for optimal expression in mammalian cells, making it an excellent marker. Luciferase sequences have been analyzed, and modified to alter the wavelength of light emitted upon interaction between the enzyme and luciferin. (See Branchini, B. R., et al., J. BIOLOGICAL CHEMISTRY 272:19359 (1997); Branchini, B. R., et al., BIOCHEMISTRY 38:13223 (1999); Branchini, B. R., et al., BIOCHEMISTRY 39:5433 (2000), Branchini, B. R., et al., BIOCHEMISTRY 40:2410 (2001); Eames, B. F., et al., in SPIE PROC. BIOMED. IMAGING: REPORTERS, DYES AND INSTRUMENTATION 3600:36 (1999); Kajiyama, N., et al., PROTEIN ENGINEERING 4:691 (1991)).

Luciferases have also been analyzed as they relate to other enzymes. For example, Conti, et al. (STRUCTRRE 4:287 (1996)) have used the crystalline structure of firefly luciferase to study the superfamily of adenylate-forming enzymes. Monsees, T., et al., (ANALYTICAL BIOCHEMISTRY 221:329 (1994) modified aminoluciferin through the addition of N-Ace-Phe to create a luminescing substrate of α-chymotrypsin.

A need exists for a highly specific reporter system for use in vivo and in vitro for monitoring biological systems, locating sites of activity, and the like. The reporter system must have means for avoiding the background found in many current systems that use fluorescence. Further, the system preferably does not interfere with the native functioning of the cells or tissues.

Additional needs in the art include a method for precisely targeting a compound, such as a drug, to a particular set of cells or tissues, where the passage of the drug across either the cell membrane or tissue boundary can be monitored in vivo.

### SUMMARY OF THE INVENTION

In one aspect of the invention, there is provided an aminoluciferyl peptide substrate having the structure of formula (I)
Ac-Ser-Lys-Leu-Gln-aLuc.

In another aspect of the invention, the aminoluciferyl peptide substrate of the present invention is used to quantify the amount of prostate specific antigen (“PSA”) in an animal subject.

In yet another aspect of the invention, there is provided a method of detecting PSA in an animal subject comprising the steps of (a) conjugating aminoluciferin with a peptide sequence that is specific for PSA to produce an aminoluciferyl peptide substrate that may only be cleaved in the presence of PSA; (b) contacting the aminoluciferyl peptide substrate with prostate cells; and (c) monitoring the cells for light emission that indicates the presence of prostate cancer cells, wherein the PSA cleaves the peptide sequence from the aminoluciferin, and light emissions result when the aminoluciferin reacts with luciferase present at the site. The prostate cancer cells of this method may be cultured in vitro and used in vivo to generate a tumor.

In a further embodiment of the invention, there is provided a method of delivering agents to specific sites in an animal species, including humans, comprising the steps of (a) conjugating aminoluciferin with an imaging agent to form an aminoluciferin-agent conjugate; (b) conjugating the aminoluciferin-agent conjugate with a peptide sequence that cannot penetrate cell membranes or tissue barriers, to produce an agent-luciferyl peptide substrate that will not penetrate cell membranes or tissue barriers; (c) injecting the animal species with the agent-luciferyl peptide substrate, wherein within the animal species, the peptide sequence is cleaved by a target enzyme on a target cell or tissue to reform the luciferin-agent conjugate; (d) monitoring the animal species for signals from the luciferin-agent conjugate that indicate passage of the luciferin-agent conjugate across the cell membrane or tissue barrier and retention of the luciferin-agent conjugate in cells or tissue, wherein the signal from the luciferin-agent facilitates localization of the luciferin-agent conjugate. The cell membranes include, without limitation, tumor cell membranes, neuronal membranes, and other cell membranes, and the tissue barriers include, without limitation, placental barriers and blood-brain barriers.

In yet another embodiment of the invention, there is provided a method of delivering therapeutic agents to specific sites in an animal species, including humans, comprising the steps of (a) conjugating luciferin with a therapeutic agent to form a luciferin-agent conjugate; (b) conjugating the luciferin-agent conjugate with a peptide sequence that cannot penetrate cell membranes or cross tissue barriers to produce an agent-luciferyl peptide substrate that will not penetrate cell membranes or tissue barriers, wherein the peptide sequence can be cleaved by a target enzyme on a target cell or tissue; (c) injecting the animal species with the agent-luciferyl peptide substrate, thereby delivering the agent-luciferyl substrate to the target cell or tissue; (d) wherein the peptide sequence is cleaved from the agent-luciferyl substrate by a target enzyme such that the luciferin-agent conjugate is reformed, wherein the therapeutic agents are delivered to the animal species upon passage of the luciferin-agent conjugate across the cell membrane or tissue barrier and retention of the luciferin-agent conjugate in the tissue. The cell membranes include, without limitation, tumor cell membranes, neuronal membranes, and other cell membranes, and the tissue barriers include, without limitation, placental barriers and blood-brain barriers.

Additional aspects, advantages and features of the invention will be set forth, in part, in the description that follows, and, in part, will become apparent to those skilled in the art upon examination of the following, or may be learned by practice of the invention.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1 shows the conversion of luciferase to oxyluciferin in the presence of ATP, O₂, and luciferase releasing light.

FIG. 2 shows the bioluminescent probe design for luciferin. FIG. 2A shows the native form with a hydroxyl group, while FIG. 2B shows aminoluciferin, with an amine group for adding peptides.

FIG. 3 shows an in vivo protease assay of the invention wherein aminoluciferin is cleaved from N-Ace-Phe-aminoluciferin in the presence of the protease chymotrypsin and subsequently catalyzed by luciferase in the presence of ATP and O₂ to release light.

FIG. 4 shows images of mice that have been injected with luciferin (FIG. 4A) and aminoluciferin (FIG. 4B).

FIG. 5 shows a schematic of the application of the invention to detecting prostate-specific antigen (PSA).

FIG. 6 shows a luminometer measurement of PSA cleavage of aLuc from Ac-Ser-Lys-Leu-Gln-aLuc.

FIG. 7 shows photon emissions from LNCaP-Clone9 and PC3M upon addition of PSA peptide-substrate (the incubation time indicates the amount of time that PSA was allowed to accumulate before the peptide-substrate was added).

FIG. 8A shows LNCaP tumors imaged in SCID mice 2 weeks post-injection with 0.1 and 1. mmole of PSA peptide substrate. FIG. 8B shows an image of two SCID mice with 8 week old LNCaP-Luc tumors.

FIG. 9 is a graph showing that mutations in the luciferase enzyme change the emissions spectrum of the enzyme.

FIG. 10 shows strategies that may be used to enhance the use of the luciferyl peptide substrates in multiplexing assays.

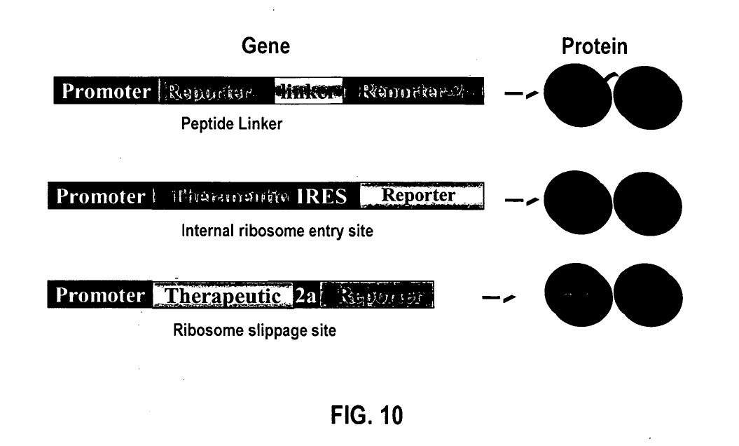

### DETAILED DESCRIPTION OF THE INVENTION

Before describing the present invention in detail, it is to be understood that this invention is not limited to particular drugs or imaging agents, and as such may vary from what is described herein. It is also to be understood that the terminology used herein is for the purpose of describing particular embodiments only, and is not intended to be limiting.

As used in this specification and the appended claims, the singular forms “a,” “an,” and “the” include plural referents unless the context clearly dictates otherwise. The use of the words “optional” and “optionally” in this specification and the appended claims indicates that the subsequently described event or circumstance may or may not occur, and that the description includes instances where said event or circumstance occurs and instances where it does not. For example, reference to an “optional member” in a formulation indicates that such a member may or may not be present, and the description includes formulations wherein a member is present and formulations wherein a member is not present.

As used herein, “analog” means a structural derivative of a parent compound that often differs from the parent by a single element. For example, aminoluciferin is an analog of luciferin because it differs only in the replacement of the hydroxyl group of luciferin with an amino group.

As used herein, “derivative” means a compound derived or obtained from another and containing essential elements of the parent substance. Thus, as used herein, because an analog is one type of a derivative, the term derivative will be meant to include analogs of the subject compound.

Conversion of Luciferin into Aminoluciferin

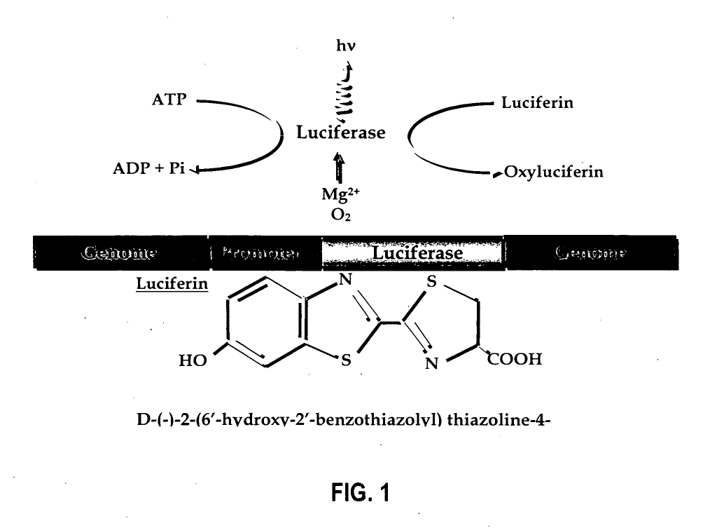

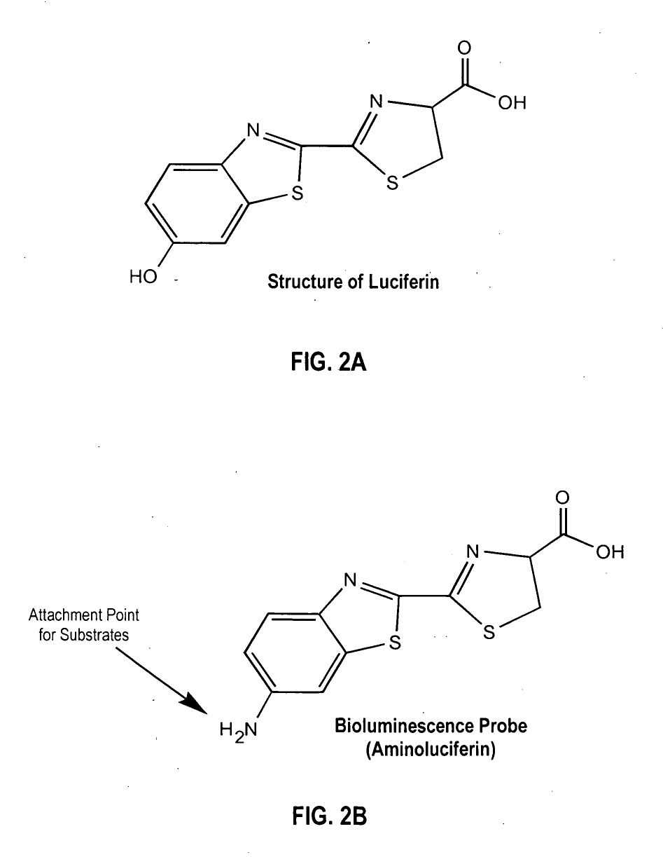

As shown in FIG. 1, luciferin [(D)-(−)-2-(6′-Hydroxy-2′-benzothiazolyl)-2-thiazoline-4-carboxylic acid] reacts with ATP in the presence of Mg²⁺/O₂ and the luciferase enzyme to produce light. As shown in FIG. 2, replacement of the hydroxyl group on luciferin with an amino group generates the luciferin analog, aminoluciferin [D-(−)-2-(6′-amino-2′-benzothiazolyl)-Δ²-thiazoline-4-carboxylic Acid], which generates light to the same extent as does luciferin when in the presence of ATP, Mg²⁺⁺/O₂ and luciferase. The preparation of aminoluciferin was first described in Katz, supra, and the synthesis of 6-aminoluciferin was described in 1994 by Monsees et al., supra. Aminoluciferin has several advantages over luciferin. For example, while aminoluciferin produces the same type and amount of light in the same manner as does luciferin, it has the advantage of being further modifiable through the addition of peptide chains to its amino group.

Protease Assays

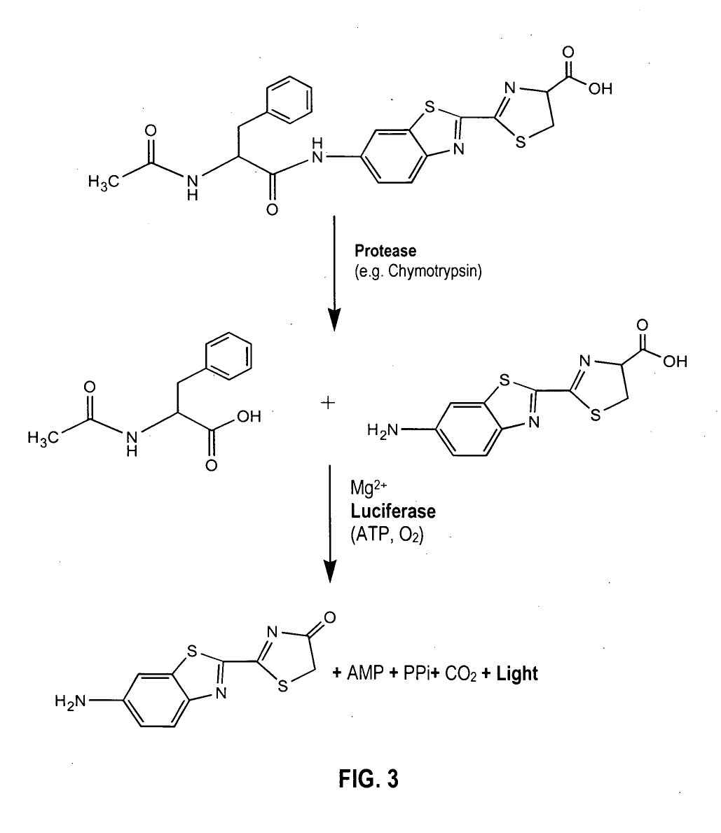

The procedure described in FIG. 2 may be used in in vivo protease assays. Naturally occurring proteases can be used to convert luciferylpeptides to aminoluciferin. These naturally occurring proteases may be associated with a physical state of the body, an organ, tissue, or cells, or may be associated with the location of particular conditions. An example of such an assay is shown in FIG. 3. There, the luciferylpeptide, N-Ac-Phe-aminoluciferin, was synthesized from a solution of 6-amino-2-cyanobenzothiazole, pursuant to the procedure set forth in Monsees et al., supra. In this form, the luciferin moiety was prevented from luminescing. Upon addition of chymotrypsin, aminoluciferin was cleaved from the luciferylpeptide. The aminoluciferin was then available for reaction with luciferase, and in the presence of ATP and O₂ in the cells, produced visible light and oxyaminoluciferin. The procedure shown in FIG. 3 was initially performed in culture and subsequently in a transgenic mouse model. This procedure can be modified by adding different compounds to the aminoluciferin, and cleaving the conjugate at the appropriate time and place with specific enzymes.

The attachment of peptide conjugates to aminoluciferin interferes with the interaction between the luciferin and the luciferase such that light is not generated when peptides are conjugated to the aminoluciferin. In this way, the luciferin may be attenuated in its bioluminescence and membrane transport functions until bioluminescence, or transport, is desired. Such bioluminescence, or transport, occurs upon cleavage of the peptide off of the aminoluciferin, releasing aminoluciferin from the conjugate. In other words, cleavage of attached peptides by proteases reactivates the protected substrate. This trait is utilized here in order to target specific sites in or on cells and/or tissues. Since the luciferase assay is extremely sensitive, this chemistry constitutes a chemical light switch that forms the basis for enzymatic assays with extraordinary sensitivity, such as those described herein.

Contag and Bachmann previously reported in vivo measurements of the luciferin-luciferase reaction in transgenic mice where the transgene contains, at least, a luciferase coding sequence, such as the luc gene. In tumor models, where the malignant cells express luciferase, the resulting bioluminescence is used as a marker to monitor tumor growth and to probe biological functions. See, Contag & Bachmann, supra. In these experiments, injection of the mice with luciferin activates the luciferin-luciferase reaction and the subsequent emission of light, i.e., photons, which can be monitored outside the body using low light imaging systems, based on charge coupled device (“CCD”) cameras.

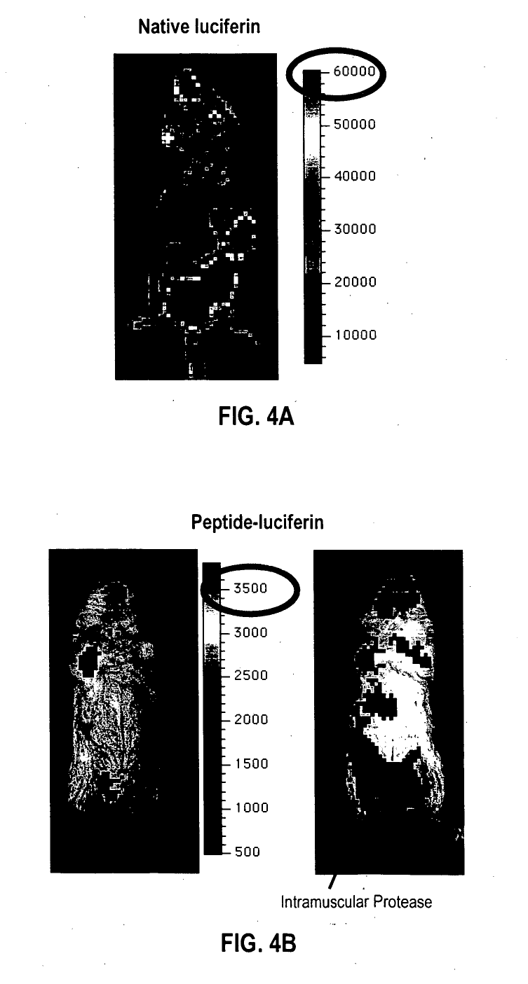

FIG. 4 shows imaging data from mice expressing the luc gene that have been injected with luciferin, aminoluciferin, and aminoluciferin/chymotrypsin. In FIG. 4A, the pictured mouse was injected with luciferin. The photograph shows that most of the tissues of this mouse emitted visible light. In FIG. 4B, the mice were injected with the modified substrate N-Ac-Phe-aminoluciferin. The photograph of the mouse on the left shows that only isolated regions of the mouse produced low level photon emission. The dramatic reduction in signal intensity (note scales on images) on the second mouse is due to the lack of freely available substrate for the luciferase reaction. The mouse pictured on the right was subjected to further injections with chymotrypsin at selected sites. Chymotrypsin is a proteolytic enzyme that can use N-Ac-Phe-aminoluciferin as a substrate. The photo of the mouse on the right shows that the chymotrypsin cleaved the bond between the phenylalanine and the aminoluciferin to release the aminoluciferin substrate. The freely available aminoluciferin was then catalyzed by luciferase to generate the photons seen in the photograph. As is readily apparent from this photograph, only the sites where the protease enzyme was injected produced detectable signals. It can thus be observed that the use of aminoluciferin substrates results in increased versatility for in vivo assays such that protease activity can be assessed in live cells in vivo or in a test tube.

In a one embodiment of the invention, peptide sequences that are conjugated to aminoluciferin are cleaved by the protease chymotrypsin or by chymotrypsin-like proteases, such as, for example, PSA, a clinical marker for prostate cancer. Activation of the aminoluciferyl peptide by chymotrypsin was demonstrated in in-vivo mouse models using a transgenic mouse where the transgene is comprised of a strong promoter (from the immediate early gene of human cytomegalovirus) to express a luciferase that had been modified for optimal expression in mammalian cells (Promega Corp.).

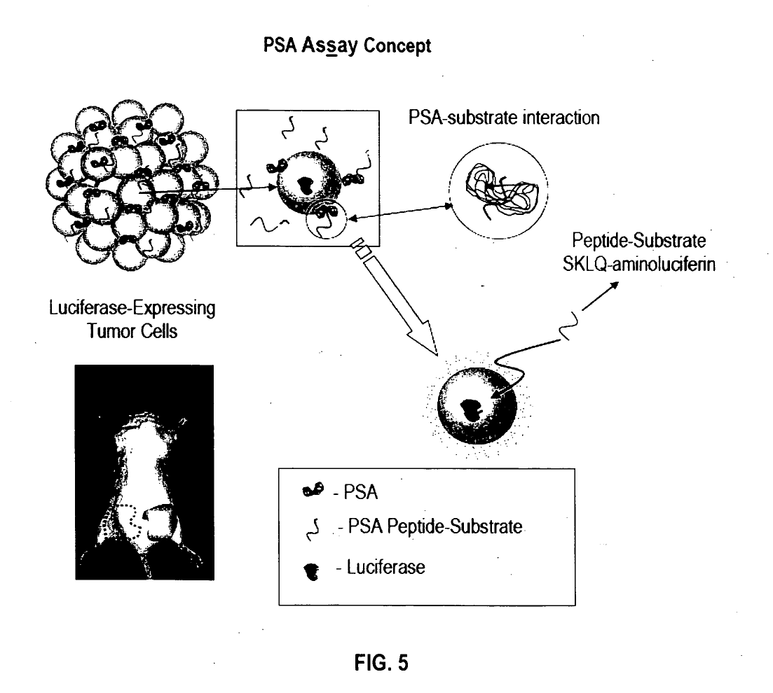

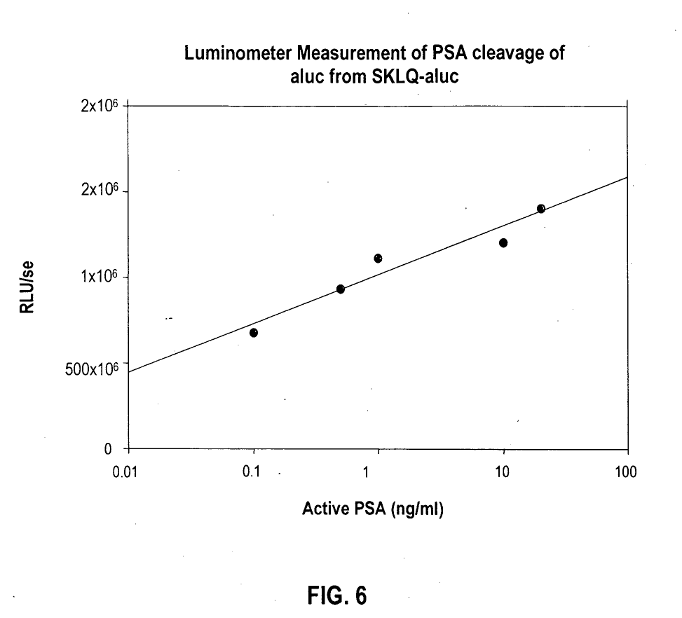

FIGS. 5 through 8 show an application of the foregoing model. This embodiment of the invention provides a method of detecting PSA in an animal subject comprising the steps of (a) conjugating luciferase-encoding aminoluciferin with a peptide sequence that is specific for the protease PSA to produce an aminoluciferyl peptide substrate that may only be cleaved in the presence of PSA; (b) contacting prostate cells with the aminoluciferyl peptide substrate; and (c) monitoring the cells for light emissions that indicate the presence of prostate cancer cells, wherein the PSA cleaves the peptide sequence from the aminoluciferin and the light emissions result when the luciferase reacts with the aminoluciferin. The prostate cells of this method may be cultured in vitro or in vivo. FIG. 5 shows a schematic of how the peptide amino-luciferyl substrate illuminates only where the cells that express luciferase also express PSA. When luciferase-expressing prostate tumors are implanted into mice, followed by injection of the amino-luciferyl substrate, light is produced (green in the Figure) only where PSA is expressed, while in a control that does not produce PSA (red dotted line shows the perimeter of the implant), no light is produced. FIG. 6 provides a graph showing increase in light in RLU/sec with increase in active PSA (ng/ml).

In addition to identifying disease conditions, such as prostate cancer, the present invention may also be used to regulate the specific delivery of agents in an animal species. Thus, another embodiment of the invention provides a method of delivering agents to specific sites in an animal species comprising the steps of (a) conjugating luciferase-encoding luciferin with an agent to form a luciferin-agent conjugate; (b) conjugating the luciferin-agent conjugate with a peptide sequence that cannot penetrate cell membranes or tissue barriers to produce an agent-luciferyl peptide substrate that will not penetrate cell membranes or tissue barriers, wherein the peptide sequence has a tag that enables the agent-luciferyl peptide substrate to be tracked by location; (c) injecting the animal species with the agent-luciferyl peptide substrate; and (d) monitoring the location of the agent-luciferyl peptide substrate by tracking the location of the tag; (e) injecting the animal species with an enzyme that will cleave the peptide sequence from the luciferin-agent conjugate; and (f) monitoring the animal species for light emissions that indicate the passage of the luciferin-agent across the cell membrane or tissue barrier, wherein the enzyme is injected in the animal when the agent luciferyl peptide substrate is identified at a desired location and the light emissions result when upon cleavage of the peptide sequence, the luciferin interacts with the luciferase enzyme. The cell membranes include, without limitation, tumor cell membranes, neuronal membranes, and other cell membranes, and the tissue barriers include, without limitation, placental barriers, and blood-brain barriers.

In a further embodiment of the invention, there is provided a method of delivering agents to specific sites in an animal species, including humans, comprising the steps of (a) conjugating luciferin with an imaging agent to form an luciferin-agent conjugate; (b) conjugating the luciferin-agent conjugate with a peptide sequence that cannot penetrate cell membranes or tissue barriers to produce an agent-luciferyl peptide substrate that will not penetrate cell membranes or tissue barriers; (c) injecting the animal species with the agent-luciferyl peptide substrate, wherein within the animal species, the peptide sequence is cleaved by a target enzyme on a target cell or tissue to reform the luciferin-agent conjugate; (d) monitoring the animal species for signals from the imaging agent that indicate passage of the luciferin-agent conjugate across the cell membrane or tissue barrier and retention of the luciferin-agent conjugate in cells or tissue, wherein the signal from the imaging agent facilitates localization of the luciferin-agent conjugate. The cell membranes include, without limitation, tumor cell membranes, neuronal membranes, and other cell membranes, and the tissue barriers include, without limitation, placental barriers, and blood-brain barriers.

In yet another embodiment of the invention, there is provided a method of delivering therapeutic agents to specific sites in an animal species, including humans, comprising the steps of (a) conjugating luciferin with a therapeutic agent to form a agent-luciferin conjugate; (b) conjugating the agent-luciferin conjugate with a peptide sequence that cannot penetrate cell membranes or cross tissue barriers to produce an agent-luciferyl peptide substrate that will not penetrate cell membranes or tissue barriers, wherein the peptide sequence can be cleaved by a target enzyme on a target cell or tissue (c) injecting the animal species with the agent-luciferyl peptide substrate; and (d) delivering the agent-luciferyl substrate to the target cell or tissue, wherein the peptide sequence is cleaved from the agent-luciferyl substrate by a target enzyme such that the luciferin-agent conjugate is reformed, wherein the therapeutic agents are delivered to the animal species upon passage of the luciferin-agent conjugate across the cell membrane or tissue barrier and retention of the luciferin-agent conjugate in the tissue. The cell membranes include, without limitation, tumor cell membranes, neuronal membranes, and other cell membranes, and the tissue barriers include, without limitation, placental barriers, and blood-brain barriers.

As indicated above, the luciferyl peptide substrates have utility in many applications, such as, for example, to identify disease states such as prostate cancer or to modulate the transport of the peptide substrates across cell membranes and tissue barriers, such as the placental barrier and the blood brain barrier mentioned above. As transport across such barriers is a major hurdle for pharmaceutical delivery the present invention provides a powerful tool for specific and regulated in vivo drug delivery. The activateable luciferyl peptide substrates of the present invention may also form the basis of molecular probes and molecular therapeutics. The use of the luciferyl peptide substrates of the present invention in this way demonstrates the potential of the present invention for developing assays for detecting pathogens, testing protease inhibitors, probing cell physiology, assessing protease activity in oncogenesis, and, as discussed above, for improving the mechanism for specific and regulated drug delivery.

As indicated above, coupling of the luciferyl peptide substrates of the present invention with detectors provides a versatile platform for imaging both in vivo and in vitro assays. For example, the luciferyl peptide substrates of the present invention may be modified with MRI, PET or SPECT tracers such as gadolinium, ¹²⁵I, ¹⁸F, etc. which can be used to visualize the location of the luciferyl peptide substrates for diagnostic and/or therapeutic applications.

The luciferyl peptide substrates of the present invention also provide a method for modifying luciferin, its analogs and derivatives, to serve as precursors for automated peptide synthesizers. In this way, the luciferin molecule, and/or analogs or derivatives thereof, are modified such that they may be readily incorporated into synthetic peptides, RNA, DNA and carbohydrates.

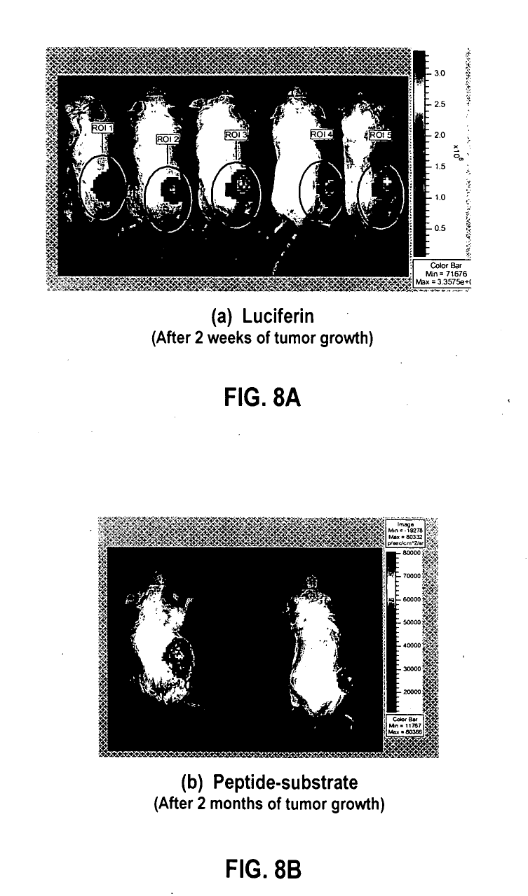

The luciferyl peptide substrates of the present invention may also be used in multiplex assays. Luciferase-luciferin partners that may be used in multiplex assays may be determined by analyzing luciferase enzyme mutants versus luciferin analogs or derivatives. Mutations in the luciferase enzyme that change the emission spectrum have been studied and synthesized by Branchini et al. See, Branchini et al. (1997, 1999, 2000, and 2001), supra. FIG. 8 shows the emission spectra of a bacterial luciferase (lux operon from soil bacterium Photorhabdus luminescens) in the blue wavelength and wild type (“WT”) and mutant emissions spectra for the North American firefly, Photinus pyralis. The mutant firefly luciferase has been modified such that the emission wavelength is shifted from 550 in its native WT state to more 612 nm in the mutant state (red mutant). A large library of luciferase mutants has already been generated and a variety of luciferin analogs have been produced. See, Conti et al., Eames et al., and Kajiyama et al., supra. Therefore, matrix analysis of mutants against luciferin analogs could be conducted to detect luciferase-luciferin partners that together further shift the emissions spectra into the red.

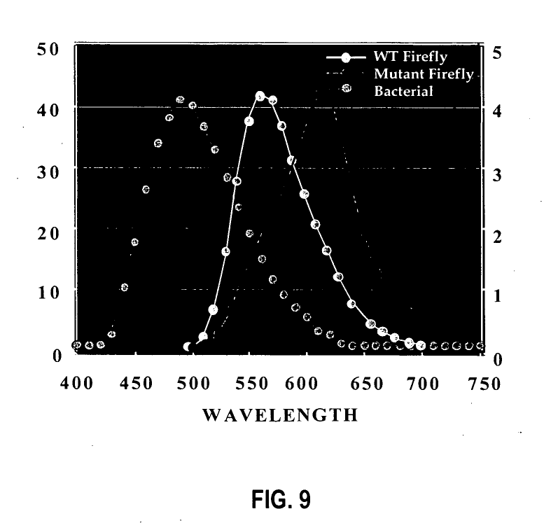

Use of multifunctional fusion genes will add to the power of such a multiplex assay. FIG. 9 shows strategies that could be used to complement the strengths of multiple genes. Two or more genes could be fused together with a flexible amino acid spacer or other strategies to generate bicistronic message. For example, coding regions could also be connected via an internal ribosome entry site (“IRES”) or a ribosome slippage site, to create a bicistronic message for co-expression of two or more reporters, or a reporter and the coding sequence of a therapeutic gene. Fluorescent and luminescent sensors could be linked to increase wavelength of emitted light and chemiluminescent resonant energy transfer, thus providing greater tissue penetration for the in vitro multiplexing assays.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents and publications mentioned herein are hereby incorporated by reference in their entireties.

The following examples are put forth so as to provide those of ordinary skill in the art with a complete disclosure and description of how to make and use the compositions of the invention. Efforts have been made to ensure accuracy with respect to numbers (e.g., amounts, temperature, etc.) but some experimental error and deviations should, of course, be allowed for. Unless indicated otherwise, parts are parts by weight, temperature is degrees centigrade and pressure is at or near atmospheric. All components were obtained commercially unless otherwise indicated.

### EXPERIMENTAL

Unless otherwise indicated, all formulations described herein were performed with commercially available products.

Abbreviations used in the Examples are set forth in Table 1.

TABLE 1

| ABBREVIATION | COMPOUND |
|---|---|
| aLuc | 6-Aminoluciferin |
| DMF | N,N,-dimethylformamide |
| LNCaP | lymph node carcinoma of the prostate |
| PC3M | prostate cancer 3-metastatic |

### EXAMPLE 1

### Aminoluciferyl Substrates and Enzymes for Use in in vivo Models

The efficacy of the luciferyl peptide substrates of the present invention in in vivo models may be determined by using the five enzymes and the eight aminoluciferyl peptide substrates shown in Table 2.

TABLE 2

| ENZYME | AMINOLUCIFERYL SUBSTRATES |
|---|---|
| Cathepsin B | 1) Bz-Arg-aLuc |
| | 2) Pyr-Phe-Leu-aLuc |
| | 3) Z-Arg-Arg-aLuc |
| PSA | 4) Ac-His-Ser-Ser-Lys-Leu-Gln-aLuc |
| | 5) Ac-Ser-Lys-Leu-Gln-aLuc |
| MMP-2 and MMP-9 | 6) Ac-Gly-Pro-Leu-Gly-Ile-Ala-Gly-Gln-aLuc |
| Thrombin | 7) Bz-Phe-Val-Arg-aLuc |
| HIV Protease | 8) Abz-Thr-Ile-Nle-aLuc-Phe-Gln-Arg-NH₂ |

The aminoluciferyl peptide substrate identified by No. 5 in Table 2 (Ac-Ser-Lys-Leu-Gln-aLuc referred to as “SKLQ-aLuc”) was synthesized as a substrate for PSA. The sequence selected for the peptide is relatively specific for PSA over other enzymes. The amount of aminoluciferin released is quantified by its instantaneous reaction with luciferase.

### EXAMPLE 2

### The Effect of PSA-Specific Aminoluciferyl Peptide Substrates on PSA Secreting Cells—an in vitro Model

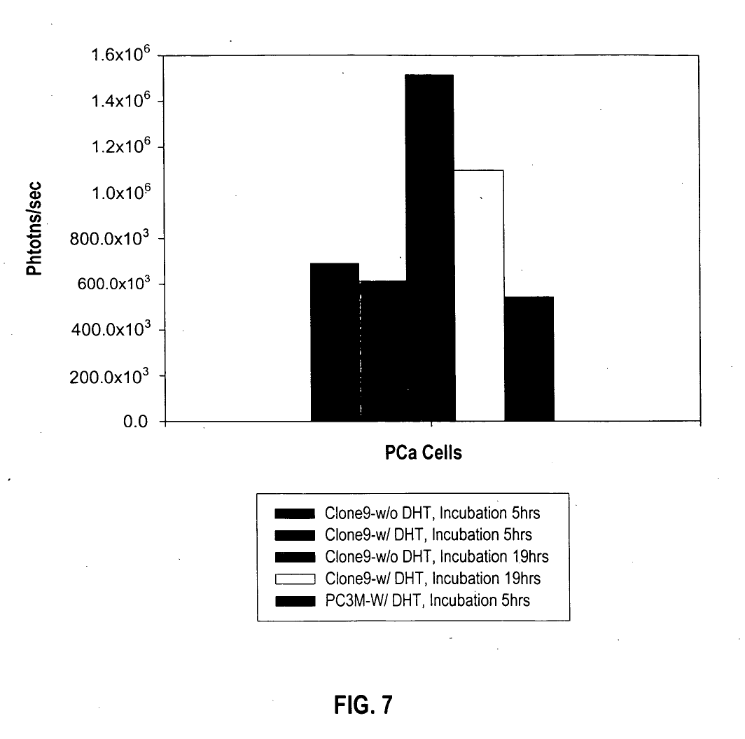

To study the relationship of the PSA-specific aminoluciferyl peptide substrates on PSA secreting cells, SKLQ-aLuc was introduced in LNCaP, a prostate cancer cell line that produces PSA, and PC3M, a prostate cancer cell line that does not produce PSA. The LNCaP cells were incubated for 5 and 19 hours, which represented two time intervals during which the cells were to synthesize PSA, and both cell lines were transfected with SKLQ-aLuc to produce luciferase. Dihydroxytestosterone (“DHT”) was used to investigate its influence on cell growth and PSA production. The cell culture media was serum-free to prevent PSA from forming complexes with various serine-protease inhibitors that are present in the serum. The results of this experiment are shown in FIG. 7, which demonstrate that the amount of aminoluciferyl peptide detected in the cells is directly proportional to the amount of PSA secreted into the media by the LNCaP cells. DHT does not appear to influence cell growth or PSA levels significantly.

### EXAMPLE 3

### The Effect of PSA-Specific Aminoluciferyl Peptide Substrates on PSA Secreting Cells—an in vivo Model

The peptides were then tested in severe compromised immune deficiency (SCID) mice that bore an LNCaP tumor implanted at a subcutaneous site. FIG. 8A shows the presence of 2 week old LNCaP tumors on the right flank of the mice. The mice were injected with luciferin to localize the tumors and confirm their presence. FIG. 8B shows light emission from these tumors 2 months after injecting the PSA-specific aminoluciferyl peptide SKLQ-aLuc. The aminoluciferin released from the cleavage of the peptide by PSA is transported across the LNCaP cell membrane and reacts with luciferase to emit light. Thus, the aminoluciferyl peptide is activated by PSA and consequently can be used to target PSA producing cells in animal models.

We claim:

1. An aminoluciferyl peptide substrate having the structure of formula (I)
Ac-Ser-Lys-Leu-Gln-aLuc. (I)

2. The aminoluciferyl peptide substrate of claim 1 used to quantify the amount of prostate specific antigen in an animal subject.

3. A method of detecting prostate specific antigen in an animal subject comprising the steps of:
(a) conjugating aminoluciferin with a peptide sequence that is specific for prostate specific antigen to produce an aminoluciferyl peptide substrate that can only be cleaved in the presence of prostate specific antigen;
(b) contacting the aminoluciferyl peptide substrate with prostate cells; and
(c) monitoring the cells for light emissions that indicate the presence of prostate cancer cells,
wherein the prostate specific antigen cleaves the peptide sequence from the aminoluciferin and the light emissions result when the aminoluciferin binds to the luciferase.

4. The method of claim 3, wherein the prostate cancer cells are cultured in vitro.

5. The method of claim 3, wherein the prostate cancer cells are localized in an animal and the method is carried out in vivo.

6. A method of delivering agents to a specific site in an animal species comprising the steps of:
(a) conjugating luciferin with an imaging agent to form a luciferin-agent conjugate;
(b) conjugating the luciferin-agent conjugate with a peptide sequence that cannot penetrate cell membranes or tissue barriers, to produce an agent-luciferyl peptide substrate that will not penetrate cell membranes or tissue barriers,
(c) injecting the animal species with the agent-luciferyl peptide substrate, wherein within the animal species, the peptide sequence is cleaved by a target enzyme on a target cell or tissue to reform the luciferin-agent conjugate;
(d) monitoring the animal species for signals from the imaging agent that indicate passage of the luciferin-agent conjugate across the cell membrane or tissue barrier and retention of the luciferin-agent conjugate in cells or tissue,
wherein the signal from the imaging agent facilitates localization of the luciferin-agent conjugate.

7. The method of claim 6, wherein the cell membrane is selected from tumor cell membrane and neuronal cell membrane.

8. The method of claim 6, wherein the tissue barrier is selected from placental barrier a blood-brain barrier.

9. A method of delivering therapeutic agents to specific sites in an animal species, including humans, comprising the steps of:
(a) conjugating luciferin with a therapeutic agent to form an agent-luciferin conjugate;
(b) conjugating the agent-luciferin conjugate with a peptide sequence that cannot penetrate cell membranes or cross tissue barriers to produce an agent-luciferyl peptide substrate that will not penetrate cell membranes or tissue barriers, wherein the peptide sequence can be cleaved by a target enzyme on a target cell or tissue;
(c) injecting the animal species with the agent-luciferyl peptide substrate; and
(d) delivering the agent-luciferyl substrate to the target cell or tissue, wherein the peptide sequence is cleaved from the agent-luciferyl substrate by a target enzyme such that the luciferin-agent conjugate is reformed,
wherein the therapeutic agent is delivered to the animal species upon passage of the luciferin-agent conjugate across the cell membrane or tissue barrier and retention of the luciferin-agent conjugate in the cell or tissue.

10. The method of claim 9, wherein the cell membrane is selected from tumor cell membrane and neuronal cell membrane.

11. The method of claim 9, wherein the tissue barrier is selected from placental barrier and blood-brain barrier.
